(19) United States
(12) Patent Application Publication
(10) Pub. No.: US 2024/0087120 A1
(43) Pub. Date: Mar. 14, 2024

(54) GEOGRAPHIC ATROPHY PROGRESSION PREDICTION AND DIFFERENTIAL GRADIENT ACTIVATION MAPS

(71) Applicant: GENENTECH, INC., South San Francisco, CA (US)

(72) Inventors: Neha Sutheekshna ANEGONDI, Fremont, CA (US); Simon Shang GAO, San Francisco, CA (US); Julia Gabriella CLUCERU, San Francisco, CA (US)

(21) Appl. No.: 18/513,106

(22) Filed: Nov. 17, 2023

Related U.S. Application Data
(63) Continuation of application No. PCT/US2022/029699, filed on May 17, 2022.
(60) Provisional application No. 63/189,679, filed on May 17, 2021.

(51) Int. Cl. G06T 7/00; G16H 50/20

(52) U.S. Cl. CPC G06T 7/0012; G16H 50/20; G06T 2207/20081

(57) ABSTRACT

A method for evaluating geographic atrophy. A set of retinal images is received. Each model of a plurality of models is trained to predict a set of geographic atrophy (GA) progression parameters for a geographic atrophy (GA) lesion using the set of retinal images. A visualization output is generated for each model of the plurality of models. The visualization output for a corresponding model of the plurality of models provides information about how the corresponding model uses the set of retinal images to predict the set of GA progression parameters.

### CROSS-REFERENCE TO RELATED APPLICATIONS

This application is a continuation of International Application No. PCT/US2022/029699, filed May 17, 2022, and entitled “Geographic Atrophy Progression Prediction And Differential Gradient Activation Maps,” which claims priority to U.S. Provisional Patent Application No. 63/189,679, entitled “Geographic Atrophy Progression Prediction and Differential Gradient Activation Maps,” filed May 17, 2021, each of which is incorporated herein by reference in its entirety.

### FIELD

This description is generally directed toward the prediction of geographic atrophy progression. More specifically, this description provides methods and systems for predicting geographic atrophy progression using various models and analyses (e.g., gradient activation map analysis, ablation analysis) performed for these models.

### BACKGROUND

Age-related macular degeneration (AMD) is a leading cause of vision loss in patients 50 years or older. Geographic atrophy (GA) is one of two advanced stages of AMD and is characterized by progressive and irreversible loss of choriocapillaris, retinal pigment epithelium (RPE), and photoreceptors. GA progression varies between patients, and currently, no widely accepted treatment for preventing or slowing down the progression of GA exists. Therefore, evaluating GA progression in individual patients may be important to researching GA and developing an effective treatment.

### SUMMARY

In one or more embodiments, a method is provided for evaluating geographic atrophy. A set of retinal images is received. Each model of a plurality of models is trained to predict a set of geographic atrophy (GA) progression parameters for a geographic atrophy (GA) lesion using the set of retinal images. A visualization output is generated for each model of the plurality of models. The visualization output for a corresponding model of the plurality of models provides information about how the corresponding model uses the set of retinal images to predict the set of GA progression parameters.

In one or more embodiments, a method is provided for evaluating geographic atrophy in a retina. A set of retinal images is received. A set of geographic atrophy (GA) progression parameters for a geographic atrophy (GA) lesion in the retina is predicted using the set of retinal images and a deep learning model. A set of gradient activation maps corresponding to the set of retinal images is generated for the deep learning model. A gradient activation map in the set of gradient activation maps for a corresponding retinal image of the set of retinal images identifies a set of regions in the corresponding retinal image that is relevant to predicting the set of GA progression parameters by the deep learning model.

In one or more embodiments, a system for managing evaluating geographic atrophy comprises a memory containing machine readable medium comprising machine executable code and a processor coupled to the memory. The processor is configured to execute the machine executable code to cause the processor to receive a set of retinal images; train each model of a plurality of models to predict a set of geographic atrophy (GA) progression parameters for a geographic atrophy (GA) lesion using the set of retinal images; and generate a visualization output for each model of the plurality of models. The visualization output for a corresponding model of the plurality of models provides information about how the corresponding model uses the set of retinal images to predict the set of GA progression parameters.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a block diagram of a lesion evaluation system 100 in accordance with various embodiments.

FIG. 2 is a flowchart of a process for evaluating geographic atrophy in accordance with various embodiments.

FIG. 3 is a flowchart of a process for evaluating geographic atrophy in accordance with various embodiments.

FIG. 4 is a flowchart of a process 400 for improving model performance in accordance with various embodiments.

FIG. 5 is a chart comparing the gradient activation maps generated for two deep learning models where the GA lesions are unifocal lesions, in accordance with one or more embodiments.

FIG. 6 is a chart comparing the gradient activation maps generated for two deep learning models where the GA lesions are multifocal lesions, in accordance with one or more embodiments.

FIG. 7 is a chart depicting exemplary ablated images in accordance with one or more embodiments.

FIG. 8 is a block diagram of a computer system in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

The ability to accurately predict geographic atrophy (GA) progression based on baseline assessments may be useful in many different scenarios. Parameters associated with GA progression include lesion growth rate and baseline lesion area. Baseline lesion area is the total area of a GA lesion (e.g., in mm²). Baseline lesion area has been shown to be an indicator of GA progression, which may be evaluated based on lesion growth rate. Lesion growth rate, which may be also referred to herein as GA lesion growth rate or growth rate, is the change in lesion area over some time period. Oftentimes, the growth rate is annualized (e.g., mm²/year).

Being able to automatically predict baseline lesion area, lesion growth rate, or both using an input retinal image and a model (e.g., deep learning model) may help improve patient screening, enrichment, and/or stratification in clinical trials where the goal is to slow GA progression, thereby allowing for improved assessment of treatment effects. Improving predictions of such GA progression parameters (e.g., lesion growth rate, baseline lesion area) may also improve clinical trial efficacy through, for example, without limitation, allowing for covariate adjustment during analysis. Covariate adjustment may be used to reduce the variance of the treatment effect estimate in the clinical trial and increase the power of the clinical trial. Additionally, in some cases, predictions of GA progression parameters may be used to understand disease pathogenesis via correlation to genotypic or phenotypic signatures.

A GA lesion can be imaged by various imaging modalities including, but not limited to, fundus autofluorescence (FAF) and optical coherence tomography (OCT). For example, fundus autofluorescence (FAF) images may be input into one or more models (e.g., one or more deep learning models) to predict baseline lesion area, lesion growth rate, or both. The FAF images may be baseline FAF images that are taken at a baseline point in time. The baseline point in time may be the beginning of the clinical trial, the time of the initial assessment, a time just prior to a first administration of treatment, a time coincident with the first administration of treatment, a same day as the first administration of treatment, or some other baseline point in time.

The embodiments described herein recognize that it may be desirable to understand how a deep learning model uses one or more baseline FAF images to predict lesion growth rate, baseline lesion area, or both. For example, it may be desirable to understand which regions or features of a baseline FAF image contribute to the lesion growth rate that is predicted. Identifying which image regions or features that are relevant to (or drive) the prediction of lesion growth rate may help identify or localize new biomarkers, gain insight into GA pathology, develop trust that the deep learning model is not focusing on spurious or irrelevant image regions or features, and/or improve the performance of the deep learning model.

Thus, the embodiments described herein provide methods and systems for evaluating the image regions or features of images (e.g., FAF images) that contribute to the prediction of GA progression parameters (e.g., lesion growth rate, baseline lesion area, etc.) by a model (e.g., a deep learning model). In one or more embodiments, various types of visualizations are used to understand how such models process their inputs.

For example, gradient activation maps can be used to indicate which regions of input images (e.g., FAF images) contribute to the final output of a deep learning model. A gradient activation map may visually identify (e.g., via color, shading, highlighting, pattern, etc.) the one or more regions in an image that were relevant to the predictions (e.g., predicted growth rate and/or predicted lesion area) made by model. Such gradient activation maps can be used to validate the deep learning model by confirming whether or not the model is focusing on non-spurious and relevant portions of the image based on what is known or expected. Further, comparing the gradient activation maps generated for different models may help in the selection of a best model for use in predicting lesion growth rate, lesion area, or both. Generating gradient activation maps and using these gradient activation maps to assess which image regions were relevant (e.g., most relevant) to the predictions made by a deep learning model may be computationally inexpensive as compared to other methods for performing such operations. In this manner, the overall time and/or computing resources needed to perform such operations may be reduced. Further, using gradient activation maps as described herein does not require annotation (e.g., by a human or other model) of image regions, thereby making the overall process more efficient and/or more accurate.

In one or more embodiments, the gradient activation maps may be used to identify modifications that can be made to a deep learning model to improve performance of the deep learning model. For example, the gradient activation maps may be used to identify new biomarkers or localize known biomarkers to thereby more narrowly tailor the focus of the deep learning model. In some cases, the gradient activation maps may be used to narrow the focus of the deep learning model to reduce the time and computing resource expenditure of the deep learning model, while maintaining a desired level of predictive accuracy.

One or more embodiments described herein use ablation analysis to directly derive the portions of a retinal image that contribute to the one or more GA progression parameters predicted by a deep learning model. An ablation analysis may include performing segmentation of a retinal image and then ablating (e.g., removing) various combinations of the segmented regions. For example, a segmentation algorithm may be used to segment out (or separately identify) a GA lesion, a rim (e.g., 500 μm-wide margin) around the GA lesion, and a background (e.g., any portion of the image not identified as the GA lesion or the rim). Various combinations of the GA lesion, the rim, and the background may be ablated from the retinal image to form an ablated image that is then fed as input into the deep learning model. Comparing the performance of the model based on different types of ablated image inputs allows a determination of which image regions or features are relevant (e.g., most relevant) to the one or more GA progression parameters predicted by a deep learning model.

The information provided by the ablation analysis may be used to validate the deep learning model and confirming whether or not the deep learning model is focusing on non-spurious and relevant portions of the retinal image based on what is known or expected. In one or more embodiments, the ablation analysis may be used to generate an output for use in improving performance of the deep learning model. For example, the ablation analysis may be used to identify new biomarkers or localize known biomarkers to thereby more narrowly tailor the focus of the deep learning model. In some cases, the ablation analysis may be used to narrow the focus of the deep learning model to reduce the time and computing resource expenditure of the deep learning model, while maintaining a desired level of predictive accuracy. In other embodiments, the results of the ablation analysis may be used to generate an output that identifies modifications that can be made to the deep learning model to improve the accuracy and/or reliability of the deep learning model.

Recognizing and taking into account the importance and utility of a methodology and system that can provide the improvements described above, the specification describes various embodiments for evaluating GA progression by predicting one or more GA progression parameters using one or more models and evaluating how these models make their predictions using gradient activation maps and/or ablation analysis. For example, the various embodiments described herein provide methods and systems for generating visualization outputs (e.g., gradient activation maps) that can be used to better understand how these models (e.g., deep learning models) use different portions of images (e.g., FAF images) to predict the growth rates of GA lesions. Further, the various embodiments described herein also provide methods and systems for using ablation analysis to validate models and/or identify image features that are relevant (e.g., most relevant) to the prediction of growth rate.

### II. Exemplary System for Geographic Atrophy (GA) Progression Prediction

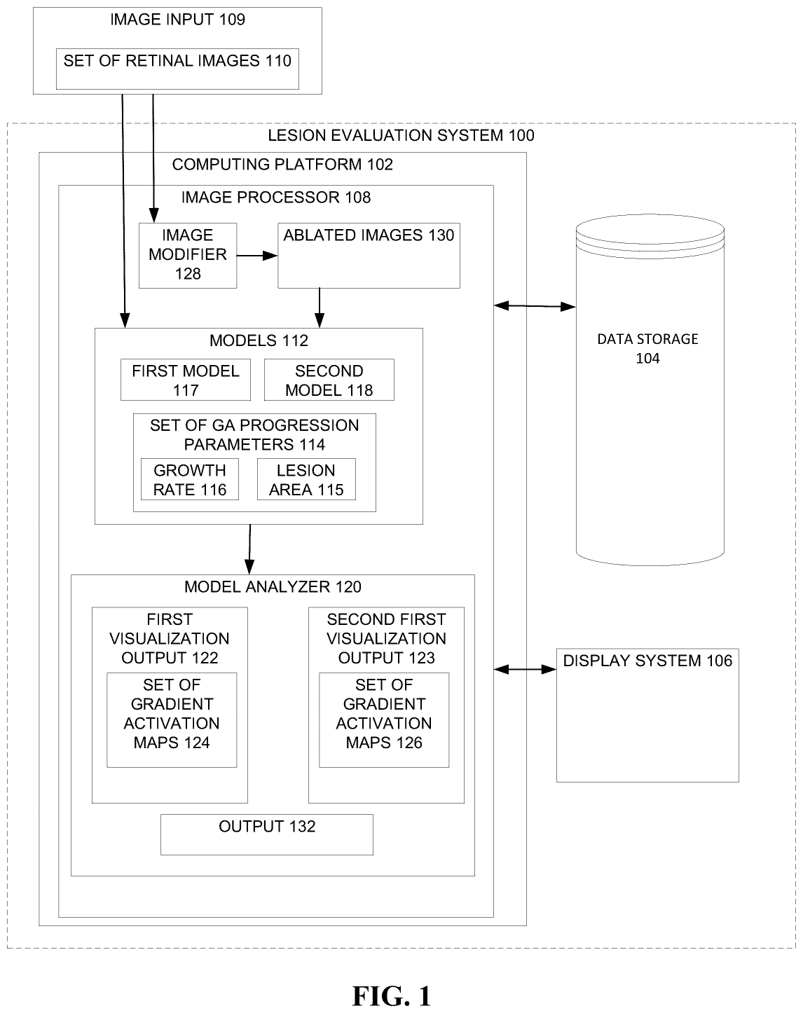

FIG. 1 is a block diagram of a lesion evaluation system 100 in accordance with various embodiments. Lesion evaluation system 100 is used to evaluate geographic atrophy (GA) lesions in the retinas of subjects. Lesion evaluation system 100 includes computing platform 102, data storage 104, and display system 106. Computing platform 102 may take various forms. In one or more embodiments, computing platform 102 includes a single computer (or computer system) or multiple computers in communication with each other. In other examples, computing platform 102 takes the form of a cloud computing platform.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

Lesion evaluation system 100 includes image processor 108, which may be implemented using hardware, software, firmware, or a combination thereof. In one or more embodiments, image processor 108 is implemented in computing platform 102.

Image processor 108 receives image input 109 for processing. Image input 109 includes one or more retinal images. In one or more embodiments, image input 109 includes one or more retinal images that are generated at a baseline or reference point in time. In some embodiments, image input 109 may be referred to as baseline image input. In one or more embodiments, image input 109 includes set of retinal images 110. Set of retinal images 110 may include, for example, without limitation, a set of fundus autofluorescence (FAF) images, a set of optical coherence tomography (OCT) images, or both. The set of FAF images may be, for example, a set of baseline FAF images. The set of OCT images may be, for example, a set of baseline OCT images. A baseline image (e.g., baseline FAF or OCT image) is an image captured at a baseline point in time. The baseline point in time may be the beginning of the clinical trial, the time of an initial assessment or initial clinic or clinical trial visit, a time just prior to a first administration of treatment, a time coincident with the first administration of treatment, a same day as the first administration of treatment, or some other baseline point in time. In other embodiments, set of retinal images 110 may include one or more other types of retinal images (e.g., color fundus (CF) photography images, near infrared (NIR) images, etc.).

Image processor 108 processes image input 109 (e.g., set of retinal images 110) using a plurality of models 112 to predict set of geographic atrophy (GA) progression parameters 114. A GA progression parameter is one that is associated with (indicates or can be used to indicate) GA progression of a GA lesion. The GA lesion may be a continuous or discontinuous lesion. For example, set of GA progression parameters 114 may include lesion area 115, growth rate 116, or both. Lesion area 115 may be a baseline lesion area for the GA lesion. Growth rate 116 (or lesion growth rate) may be the change in lesion area over a defined period of time. Growth rate 116 may be annualized (e.g., mm²/year).

Each of models 112 may be implemented in any of a number of different ways including, for example, without limitation, using one or more deep learning models. Models 112 include, for example, a first model 117 and a second model 118. In one or more embodiments, each of first model 117 and second model 118 includes a deep learning model. The deep learning model of first model 117, second model 118, or both may include, for example, without limitation, any number of or combination of neural networks. In one or more embodiments, the deep learning model may include a convolutional neural network (CNN) system that includes one or more neural networks. Each of these one or more neural networks may itself be a convolutional neural network. In some cases, the deep learning model includes multiple subsystems, each including one or more neural networks.

In one or more embodiments, image processor 108 includes model analyzer 120. Model analyzer 120 may be used to generate a visualization output for each of models 112. For example, model analyzer 120 may generate first visualization output 122 for first model 117 and second visualization output 123 for second model 118.

First visualization output 122 may include a set of visualizations for set of retinal images 110. For example, first visualization output 122 may include a visualization for each image of set of retinal images 110. First visualization output 122 provides information about how first model 117 uses set of retinal images 110 to predict set of GA progression parameters 114. Second visualization output 123 may include a set of visualizations for set of retinal images 110. For example, second visualization output 123 may include a visualization for each image of set of retinal images 110. Second visualization output 123 provides information about how second model 118 uses set of retinal images 110 to predict set of GA progression parameters 114.

In one or more embodiments, first visualization output 122 includes set of gradient activation maps 124 for set of retinal images 110 and second visualization output 123 includes set of gradient activation maps 126 for set of retinal images 110. Each gradient activation map of set of gradient activation maps 124 associated with first model 117 and set of gradient activation maps 126 associated with second model 118 may be generated using a gradient-weighted activation mapping technique for a corresponding image of set of retinal images 110. Each gradient activation map indicates the set of regions in the corresponding retinal image that contributed to the set of GA progression parameters 114 predicted by the respective associated model (e.g., first model 117 or second model 118).

For example, a gradient activation map may visually identify (e.g., via color, shading, highlighting, pattern, etc.) the one or more regions in an image that were relevant to the prediction of set of GA progression parameters 114 by the corresponding model. Further, coloring, shading, highlighting, pattern, text and/or numerical labels, other types of indicators, or a combination thereof may be used to indicate a degree of relevancy. As one example, a range of colors between red, orange, yellow, and green to blue may be used to visually identify the one the one or more regions in an image that were relevant to the prediction of set of GA progression parameters 114 by the model and their degree of relevancy. For example, a red color may be used to identify the one or more regions that had the highest degree of relevancy to the prediction of set of GA progression parameters 114, while a blue color may be used to identify the one or more regions that had the lowest degree of relevancy.

In one or more embodiments, first visualization output 122 may be used to validate first model 117, second visualization output 123 may be used to validate second model 118, or both. For example, first visualization output 122 may be used to evaluate whether the portion of the image (e.g., the one or more regions of the image) identified as being relevant to the prediction of set of GA progression parameters 114 by first model 117 align with what is expected. Similarly, second visualization output 123 may be used to evaluate whether the portion of the image (e.g., the one or more regions of the image) identified as being relevant to the prediction of set of GA progression parameters 114 by second model 118 align with what is expected.

In one or more embodiments, first visualization output 122 and second visualization output 123 may be used to determine whether any adjustments should be made to first model 117 and second model 118, respectively. For example, if first visualization output 122 identifies that the regions of the image most relevant to the prediction of set of GA progression parameters 114 by first model 117 are different from what is expected, model analyzer 120 may generate an output that indicates that an adjustment(s) should be made to first model 117. This adjustment(s) may include, for example, without limitation, retraining first model 117, changing the layers used in first model 117, modifying the architecture of first model 117, combining first model 117 with another model, or a combination thereof. Second model 118 may be similarly evaluated using second visualization output 123.

In some embodiments, first visualization output 122 generated for first model 117 may be compared to second visualization output 123 generated for second model 118 to determine similarities and/or differences in how first model 117 and second model 118 predict set of GA progression parameters 114. For example, set of gradient activation maps 124 and set of gradient activation maps 126 may be compared to determine whether the same or different regions were most relevant to the predictions made by first model 117 and second model 118.

The comparison may enable selection of a best model for use in predicting set of GA progression parameters 114. For example, model analyzer 120 may be used to generate first visualization output 122 and second visualization output 123 after first model 117 and second model 118, respectively, have been trained and tested for the prediction of set of GA progression parameters 114 based on set of retinal images 110, which may include a plurality of training baseline FAF images. These two visualization outputs may be used to determine whether one model is better suited relative to the other model for a particular type of GA lesion, whether the models perform similarly for the same types of GA lesions, etc. The information provided by these visualization outputs may then be used to determine which model to select for use in predicting set of GA progression parameters 114 for a particular subject or group of subjects.

In one or more embodiments, image processor 108 includes image modifier 128. Image modifier 128 processes image input 109 to generate a plurality of ablated images 130 that are fed as input into one or more of models 112. Analyzing the performance of models 112 using these the ablated images 130 may help identify or localize new biomarkers, gain insight into GA pathology, and develop trust that the models 112 are not focusing on spurious or irrelevant image regions or features (e.g., focusing solely the background).

The ablated images 130 may be generated in various ways. For example, an ablated image may be formed by assigning the pixels identified as corresponding to one or more selected regions (e.g., a GA lesion, a rim, a background) as black. These selected regions may be identified using, for example, a segmentation algorithm. In one example, an ablated image is formed by blacking out the pixels of the rim and the background such that the ablated image is an image of just the GA lesion (i.e., a lesion retained image). In another example, an ablated lesion is formed by blacking out the pixels of the GA lesion and the background such that the ablated image is an image of just the rim (e.g., a rim retained image). In yet another example, an ablated lesion is formed by blacking out the pixels of the GA lesion and the rim such that the ablated image is an image of just the background (e.g., a background retained image). In still yet another example, an ablated lesion is formed by blacking out the pixels of the GA lesion such that the ablated image is an image of the rim and the background (e.g., a rim and background retained image).

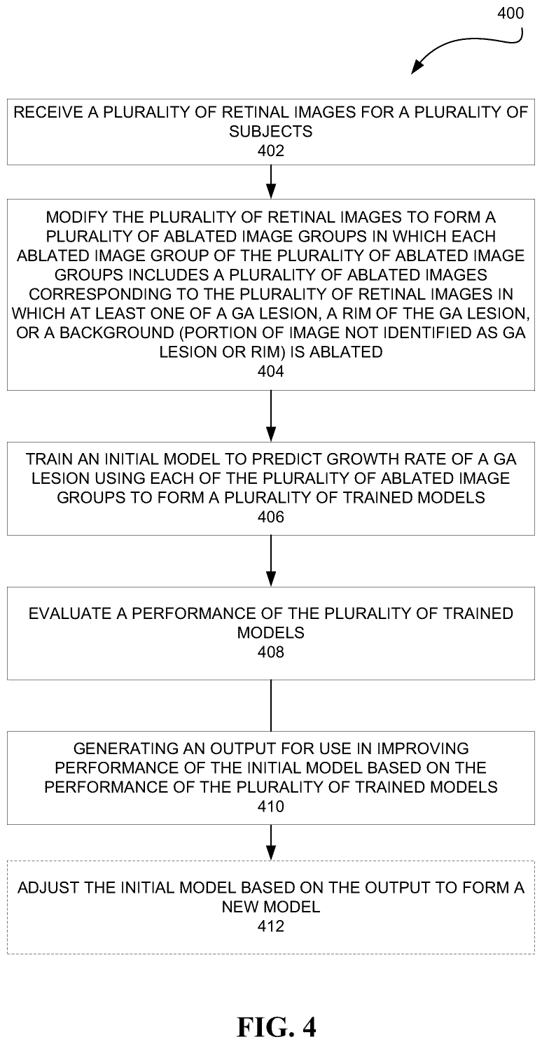

Different groupings of ablated images may be used to train, for example, first model 117 to form different trained models. The performance of these different trained models may be evaluated using, for example, model analyzer 120. Model analyzer 120 may evaluate performance with respect to accuracy, precision, reliability, a coefficient of determination (r 2), one or more other metrics, or a combination thereof. Examples of how this training and evaluation may be performed are described in greater detail with respect to FIG. 4 below. Evaluating the performance of these different trained models may help identify or localize biomarkers, gain insight into GA pathology, develop trust that first model 117 is not focusing on spurious or irrelevant image regions or features, and/or improve the performance of first model 117.

In one or more embodiments, model analyzer 120 may be used to generate output 132 based the evaluation of the performance of the different trained models formed using the ablation techniques described above and/or based on first visualization output 122 and second visualization output 123. Output 132 may, for example, identify new biomarkers and/or a localized set of biomarkers that can be used to help narrow the focus of one or more of models 112. Output 132 may, for example, identify information that provides insight into GA pathology. Output 132 may, for example, indicate whether a model of models 112 can be validated. Output 132 may, for example, confirm whether or not a model of models 112 is focusing on non-spurious and relevant image regions or features. Output 132 may, for example, identify one or more modifications that can be made to a model of models 112 to improve the performance of that model.

### III. Exemplary Methods for Evaluating GA Lesions and Models that Predict GA Lesion Growth Rate

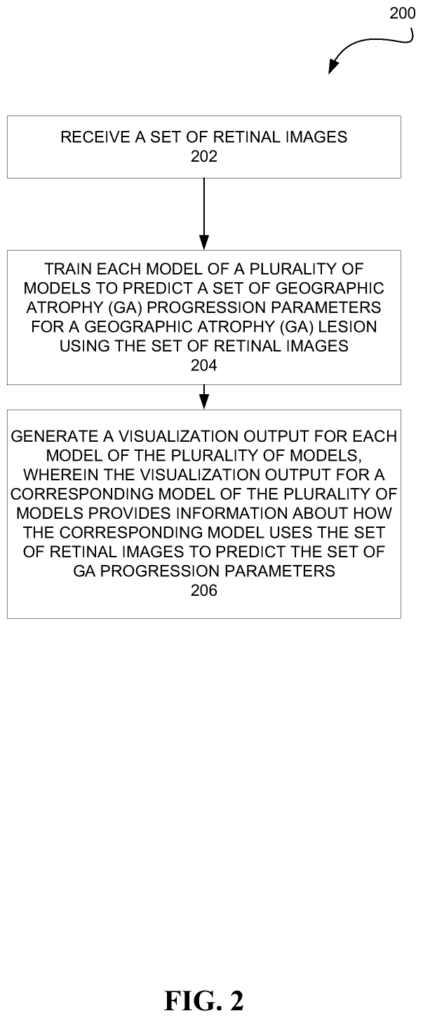

FIG. 2 is a flowchart of a process 200 for evaluating a geographic atrophy lesion in accordance with various embodiments. In various embodiments, process 200 is implemented using the lesion evaluation system 100 described in FIG. 1. In particular, process 200 may be used to predict one or more GA progression parameters.

Step 202 includes receiving a set of retinal images. The set of retinal images may be one example of an implementation for set of retinal images 110 in FIG. 1. The set of retinal images may include a set of FAF images, a set of OCT images, or both. In one or more embodiments, the set of retinal images includes a collection of baseline FAF images for a plurality of subjects that have been diagnosed with geographic atrophy or, in some cases, a precursor stage to geographic atrophy.

Step 204 includes training each model of a plurality of models to predict a set of geographic atrophy (GA) progression parameters for a geographic atrophy (GA) lesion using the set of retinal images. The plurality of models may include, for example, without limitation, a plurality of deep learning models. The plurality of models in step 204 may be one example of an implementation for models 112 in FIG. 1. As one example, the plurality of models may include a first deep learning model comprised of one or more convolutional neural networks and a second deep learning model comprised of one or more convolutional neural networks. The set of GA progression parameters may be one example of an implementation for set of GA progression parameters 114 in FIG. 1. The set of GA progression parameters may include growth rate, baseline lesion area, or both.

Step 206 generating a visualization output for each model of the plurality of models, wherein the visualization output for a corresponding model of the plurality of models provides information about how the corresponding model uses the set of retinal images to predict the set of GA progression parameters. For example, step 206 may include generating a gradient activation map for a corresponding retinal image in the set of retinal images for the corresponding model. The gradient activation map indicates (e.g., visually identifies) a set of regions in the corresponding retinal image that contributed to the set of GA progression parameters predicted by the corresponding model for the GA lesion. For example, the gradient activation map identifies the one or more regions of the retinal image that were relevant to (or that drove) the prediction of the set of GA progression parameters. Further, the gradient activation map may visually identify the degree of relevancy of these one or more regions to the prediction of the set of GA progression parameters.

In one or more embodiments, the visualization outputs generated for the different models may be used to validate these models. For example, one of the plurality of models may be a deep learning model. The visualization output generated for this deep learning model may be used to validate the deep learning model and confirm that the deep learning model is focusing on non-spurious, relevant portions of the relevant image to predict the set of GA progression parameters. In some cases, a comparison of the different visualization outputs generated for different models may be performed to select a best model for predicting the set of GA progression parameters.

In one or more embodiments, one of the plurality of models may be modified to form a new model based on the visualization output generated for that model to thereby improve a performance of the model. Performance of the model may be measured with respect to accuracy, precision, reliability, a time spent generating the prediction, an amount of computing resources utilized to generate the prediction, a coefficient of determination, or a combination thereof.

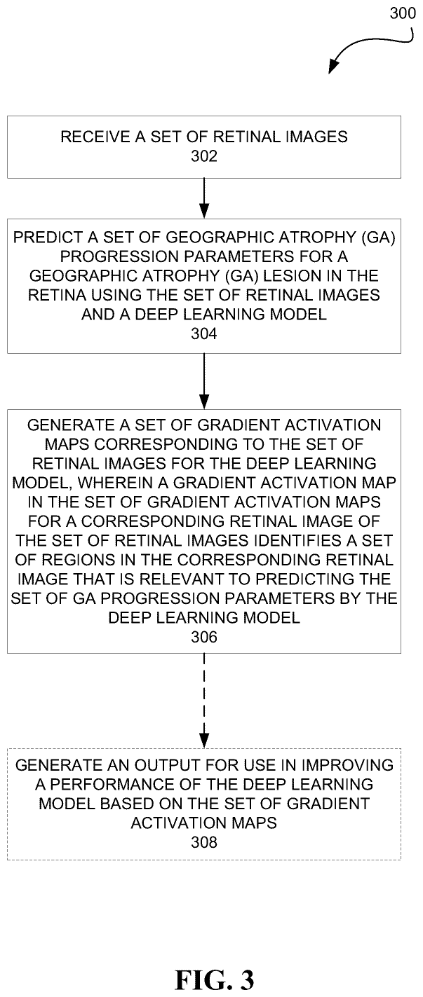

FIG. 3 is a flowchart of a process 300 for evaluating a geographic atrophy lesion in accordance with various embodiments. In various embodiments, process 300 is implemented using the lesion evaluation system 100 described in FIG. 1. In particular, process 300 may be used to predict one or more GA progression parameters.

Step 302 includes receiving a set of retinal images. The set of retinal images may be one example of an implementation for set of retinal images 110 in FIG. 1. In one or more embodiments, the set of retinal images may belong to a single subject that has been diagnosed with geographic atrophy or, in some cases, a precursor stage to geographic atrophy. For example, the set of retinal images may include a set of baseline FAF images, a set of baseline OCT images, or both for the same retina of a subject. These baseline images may include images captured for the same or substantially same (e.g., within the same hour, within the same day, within the same 1-3 days, etc.) point or points in time.

Step 304 includes predicting a set of geographic atrophy (GA) progression parameters for a geographic atrophy (GA) lesion in the retina using the set of retinal images and a deep learning model. The set of GA progression parameters comprises at least one of a growth rate for the GA lesion or a baseline lesion area for the GA lesion.

Step 306 includes generating a set of gradient activation maps corresponding to the set of retinal images for the deep learning model, wherein a gradient activation map in the set of gradient activation maps for a corresponding retinal image of the set of retinal images identifies a set of regions in the corresponding retinal image that is relevant to predicting the set of GA progression parameters by the deep learning model. The gradient activation map may, for example, visually identify a degree of relevancy of the one or more regions in the retinal image using coloring, shading, highlighting, pattern, text and/or numerical labels, other types of indicators, or a combination thereof.

Process 300 may optionally include step 308, which includes generating an output for use in improving a performance of the deep learning model based on the set of gradient activation maps. The output may, for example, identify new biomarkers and/or a localized set of biomarkers that can be used to help narrow the focus of the deep learning model, identify information that provides insight into GA pathology and that can be used to modify the deep learning model, indicate whether the deep learning can be validated, confirm whether or not the deep learning model is focusing on non-spurious and relevant image regions or features, identify one or more modifications that can be made to the deep learning model to improve the performance of that model, or a combination thereof.

FIG. 4 is a flowchart of a process 400 for improving model performance in accordance with various embodiments. In various embodiments, process 400 is implemented using the lesion evaluation system 100 described in FIG. 1.

Step 402 includes receiving a plurality of retinal images for a plurality of subjects. This plurality of retinal images may be one example of an implementation for set of retinal images 110 in FIG. 1. The plurality of retinal images may include, for example, without limitation, baseline FAF images for the subjects. These subjects may be persons who were diagnosed with GA or a precursor stage of GA and for who GA progression information (e.g., growth rate of GA lesion) is known. In one or more embodiments, each retinal image of the plurality of retinal images captures a GA lesion in the retina of a corresponding subject.

Step 404 includes modifying the plurality of retinal images to form a plurality of ablated image groups in which each ablated image group of the plurality of ablated image groups includes a plurality of ablated images corresponding to the plurality of retinal images in which at least one of a GA lesion, a rim of the GA lesion, or a background (portion of image not identified as GA lesion or rim) is ablated. The rim may be defined as, for example, a 500 μm-wide border surrounding the GA lesion. In other examples, the rim may be defined as the surrounding border having a width selected between 250 μm and 750 μm.

In one or more embodiments, ablating a selected portion or region of an image is performed by blacking out the pixels corresponding to the ablated portion or region. For example, step 404 may include segmenting the plurality of retinal images using a segmentation algorithm to identify the portion of the image that represents the GA lesion, the portion of the image that represents the rim of the GA lesion, and the background in each retinal image (portion of image not identified as GA lesion or rim). Ablating out a portion of the image may include assigning the pixels identified as representing the that portion to a value of black (e.g., a pixel value of zero). For example, the rim of the GA lesion may be ablated by assigning those pixels segmented out as representing the GA lesion to a pixel value of zero.

The plurality of ablated image groups formed in step 404 may be formed by ablating one or more different portions of the retinal images. For example, an ablated image group may be a lesion retained image group comprised of a plurality of lesion retained images in which the rim of the GA lesion and the background are ablated such that only the GA lesion of the original retinal image is retained. An ablated image group may be a lesion rim image group comprised of a plurality of rim retained images in which the GA lesion and the background are ablated such that only the rim of the GA lesion of the original retinal image is retained. An ablated image group may be a background retained image group comprised of a plurality of background retained in which the GA lesion and the rim of the GA lesion are ablated such that only the background of the original retinal image is retained.

Another ablated image group may be a lesion ablated image group comprised of a plurality of lesion ablated images in which the GA lesion is ablated such that the rim of the GA lesion and the background of the original retinal image are retained. Yet another ablated image group may be a rim ablated image group comprised of a plurality of rim ablated images in which the rim of the GA lesion is ablated such that the GA lesion and the background of the original retinal image are retained. Still another ablated image group may be a background ablated image group comprised of a plurality of background ablated images in which the background is ablated such that the GA lesion and the rim of the GA lesion of the original retinal image are retained. In this manner, different portions or combinations of portions of a retinal image may be ablated to form an ablated image.

In some embodiments, step 404 further includes shuffling the pixel values within whatever portion of the original retinal image is retained to form an ablated image. This shuffling may be a randomly-performed repositioning of the pixel values amongst the pixels included in the retained portion of the retinal image. For example, an ablated image may be a lesion shuffled image in which the portion of the retinal image identified as the GA lesion is retained and the pixel values of pixels within this portion are shuffled. This shuffling retains the intensity information associated with this portion of the retinal image but removes textural information (e.g., what areas of this portion are brighter than others). An ablated image may be a rim shuffled image in which the portion of the retinal image identified as the rim of the GA lesion is retained with the pixel values of this portion being shuffled. An ablated image may be a background shuffled image in which the portion of the retinal image identified as background is retained with the pixel values of this portion being shuffled. Accordingly, in some embodiments, the plurality of ablated image groups may include a lesion shuffled image group, a rim shuffled image group, a background shuffled image group, a lesion and rim shuffled image group, a lesion and background shuffled image group, a rim and background shuffled image group, or a combination thereof.

Step 406 includes training an initial model to predict growth rate of a GA lesion using each of the plurality of ablated image groups to form a plurality of trained models. The initial model may be, for example, a deep learning model and may include one or more neural networks. A trained model may be formed for each ablated image group of the plurality of ablated image groups. For example, the initial model may be trained and tested using a first ablated image group of the plurality of ablated image groups to form a first trained model. As another example, the initial model may be trained and tested using a second ablated image group of the plurality of ablated image groups to form a second trained model.

Step 408 includes evaluating a performance of the plurality of trained models. Performance may be analyzed with respect to any number of metrics including, for example, without limitation, accuracy, precision, a coefficient of determination (r²), a time spent by the trained model to analyze a single ablated image to predict growth rate as compared to a time spent by the initial model to analyze the corresponding original retinal image, an amount of computing resources spent by the trained model to analyze a single ablated image to predict growth rate as compared to the amount of computing resources spent by the initial model to analyze the corresponding original retinal image, one or more other types of metrics, or a combination thereof.

Step 410 includes generating an output for use in improving performance of the initial model based on the performance of the plurality of trained models. Step 410 may be performed in various ways. In one or more embodiments, the output may be an identification of the ablated image group corresponding to the trained model that had the best performance. For example, the trained model corresponding to a rim retained image group may be identified as having the best performance. In this example, the output may identify the rim of the GA lesion as most relevant to the prediction of growth rate. The output may further identify one or more biomarkers associated with this rim region with an indication that focusing on these one or more biomarkers may improve model performance with respect to speed and computing resources utilized. In this manner, identifying the rim of the GA lesion as most relevant to the prediction of growth rate may help localize the biomarkers of interest.

Process 400 may optionally include step 412, which includes adjusting the initial model based on the output to form a new model. In one or more embodiments, adjusting the initial model includes narrowing the biomarkers analyzed by the initial model to those associated with the region (e.g., GA lesion, rim of the GA lesion, or background) of the retinal identified as being most relevant to predicting growth rate. In some embodiments, adjusting the initial model includes integrating a supplemental model (which may itself include one or more algorithms or models) as part of the initial model to form the model or combining the supplemental model with the initial model to form the new model. The supplemental model may, for example, be used to segment an input retinal image and form an ablated image based on this segmentation. The new model uses the ablated image to predict growth rate, which may be faster and/or consume fewer computing resources than using the non-ablated retinal image to predict growth rate.

### IV. Exemplary Visualization Outputs and Ablated Images

A. Exemplary Visualization Outputs Generated for Two Deep Learning Models

An experiment was conducted in which two different deep learning models were trained and tested using retinal images for a plurality of subjects. These retinal images were baseline FAF images, each of which captured a GA lesion which may have been a unifocal GA lesion or a multifocal lesion. The first deep learning model and the second deep learning model were both used to predict lesion growth rate for the GA lesions. Visualization outputs were generated for these two deep learning models. Specifically, gradient activation maps were generated for the two deep learning models to provide information about what portions of each retinal image were ultimately relevant to the growth rate predicted by the corresponding deep learning model.

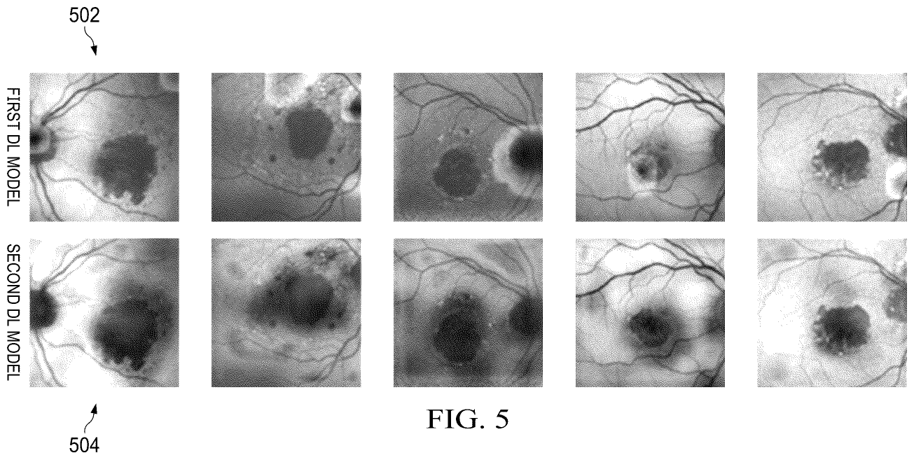

FIG. 5 is a chart comparing the gradient activation maps generated for the two deep learning models in accordance with one or more embodiments. In FIG. 5, first group of gradient activation maps 502, which was generated for the first deep learning model (1^{st} DL Model), is one example of an implementation for at least a portion of set of gradient activation maps 124 in FIG. 1. Second group of gradient activation maps 504, which was generated for the second deep learning model (2^{nd} DL Model), is one example of an implementation for at least a portion of set of gradient activation maps 126 in FIG. 1.

The five gradient activation maps in first group of gradient activation maps 502 and the five gradient activation maps in second group of gradient activation maps 504 were generated for the same group of five retinal images, each of which captured a unifocal GA lesion. Comparing first group of gradient activation maps 502 with second group of gradient activation maps 504 reveals that for retinal images of unifocal GA lesions, different portions of these retinal images were relevant to the first deep learning model as compared to the second deep learning model.

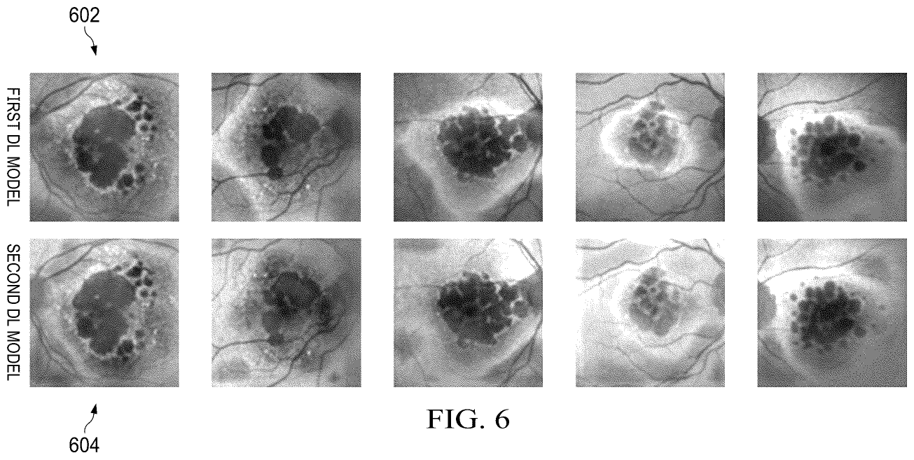

FIG. 6 is a chart comparing the gradient activation maps generated for the two deep learning models in accordance with one or more embodiments. In FIG. 6, first group of gradient activation maps 602, which was generated for the first deep learning model (1^{st} DL Model), is one example of an implementation for at least a portion of set of gradient activation maps 124 in FIG. 1. Second group of gradient activation maps 604, which was generated for the second deep learning model (2^{nd} DL Model), is one example of an implementation for at least a portion of set of gradient activation maps 126 in FIG. 1.

The five gradient activation maps in first group of gradient activation maps 602 and the five gradient activation maps in second group of gradient activation maps 604 were generated for the same group of five retinal images, each of which captured a multifocal GA lesion. Comparing first group of gradient activation maps 602 with second group of gradient activation maps 604 reveals that for retinal images of multifocal GA lesions, similar portions of these retinal images were relevant to both the first deep learning model as compared to the second deep learning model.

B. Exemplary Ablated Images for Ablation Analysis

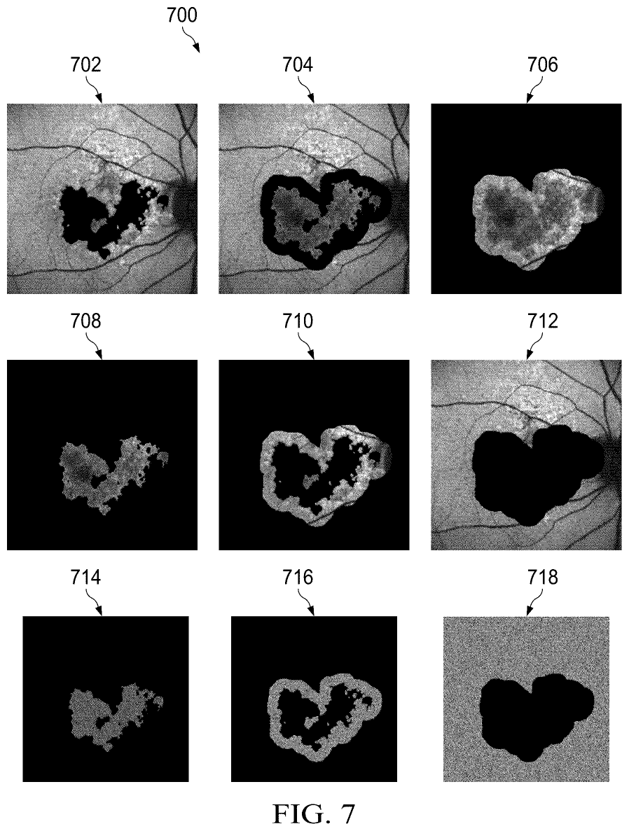

FIG. 7 is a chart depicting exemplary ablated images in accordance with one or more embodiments. Each of ablated images 700 may be one example of an implementation for an ablated image of ablated images 130 in FIG. 1. Ablated images 700 include a lesion ablated image 702, a rim ablated image 704, a background ablated image 706, a lesion retained image 708, a rim retained image 710, a background retained image 712, a lesion shuffled image 714, a rim shuffled image 716, and a background shuffled image 718.

### V. Computer Implemented System

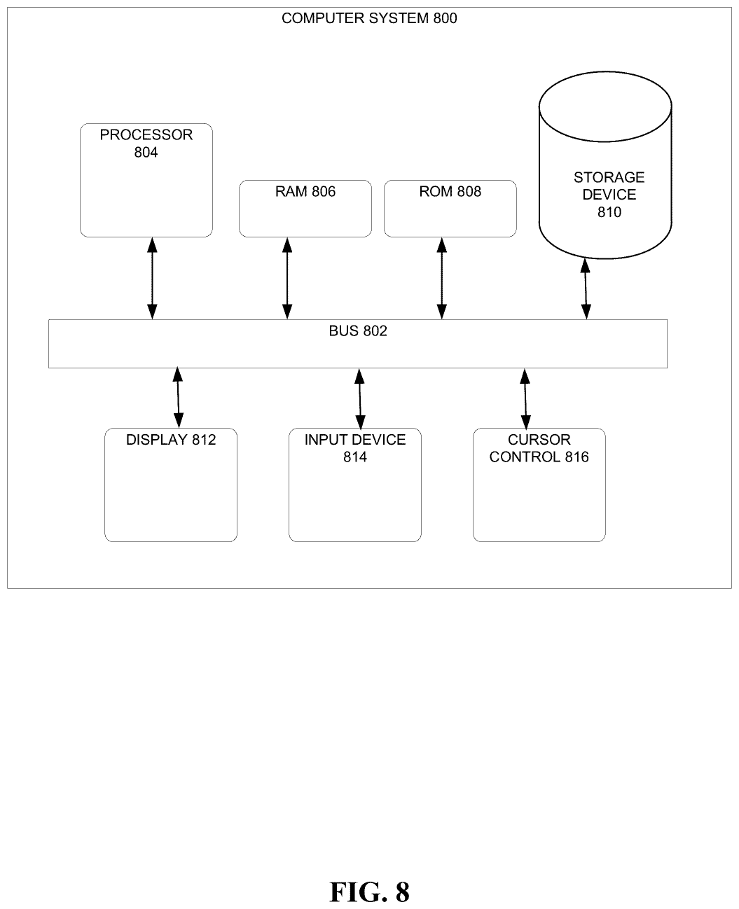

FIG. 8 is a block diagram of a computer system in accordance with various embodiments.

Computer system 800 may be an example of one implementation for computing platform 102 described above in FIG. 1. In one or more examples, computer system 800 can include a bus 802 or other communication mechanism for communicating information, and a processor 804 coupled with bus 802 for processing information. In various embodiments, computer system 800 can also include a memory, which can be a random-access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT) or liquid crystal display (LCD), for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y and z) cursor movement are also contemplated herein.

Consistent with certain implementations of the present teachings, results can be provided by computer system 800 in response to processor 804 executing one or more sequences of one or more instructions contained in RAM 806. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 810. Examples of volatile media can include, but are not limited to, dynamic memory, such as RAM 806. Examples of transmission media can include, but are not limited to, coaxial cables, copper wire, and fiber optics, including the wires that comprise bus 802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

### VI. Exemplary Context and Definitions

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

In addition, as the terms “on,” “attached to,” “connected to,” “coupled to,” or similar words are used herein, one element (e.g., a component, a material, a layer, a substrate, etc.) may be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person undergoing treatment, a person undergoing anti-cancer therapies, a person being monitored for remission or recovery, a person undergoing a preventative health analysis (e.g., due to their medical history), or any other person or patient of interest. In various cases, “subject” and “patient” may be used interchangeably herein.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” may mean sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, may mean different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming.

As used herein, an “artificial neural network” or “neural network” (NN) may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways; when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “lesion” may be a region in an organ or tissue that has suffered damage via injury or disease. This region may be a continuous or discontinuous region. For example, as used herein, a lesion may include multiple regions. A geographic atrophy (GA) lesion may be a region of the retina that has suffered chronic progressive degeneration. As used herein, a GA lesion may include one lesion (e.g., one continuous lesion region) or multiple lesions (e.g., discontinuous lesion region comprised of multiple, separate lesions).

As used herein, a “lesion area” may mean the total area covered by a lesion, whether that lesion be a continuous region or a discontinuous region.

As used herein, “longitudinal” may mean over a period of time. The period of time may be in days, weeks, months, years, or some other measure of time.

As used herein, a “growth rate” corresponding to a GA lesion may mean a longitudinal change in the lesion area of the GA lesion. This growth rate may also be referred to as a GA growth rate.

### VII. Recitation of Embodiments

Embodiment 1. A method for evaluating geographic atrophy, the method comprising: receiving a set of retinal images; training each model of a plurality of models to predict a set of geographic atrophy (GA) progression parameters for a geographic atrophy (GA) lesion using the set of retinal images; and generating a visualization output for each model of the plurality of models, wherein the visualization output for a corresponding model of the plurality of models provides information about how the corresponding model uses the set of retinal images to predict the set of GA progression parameters.

Embodiment 2. The method of embodiment 1, wherein the generating comprises: generating a gradient activation map for a corresponding retinal image in the set of retinal images for the corresponding model, wherein the gradient activation map indicates a set of regions in the corresponding retinal image that contributed to the set of GA progression parameters predicted by the corresponding model for the GA lesion.

Embodiment 3. The method of embodiment 1 or embodiment 2, wherein the plurality of models includes a deep learning model and further comprising: validating the deep learning model using the visualization output generated for the deep learning model.

Embodiment 4. The method of any one of embodiments 1-3, wherein the plurality of models includes a first deep learning model and a second deep learning model further comprising: performing a comparison of the visualization output generated for the first deep learning model with the visualization output generated for the second deep learning model.

Embodiment 5. The method of embodiment 4, further comprising: selecting either the first deep learning model or the second deep learning model as a best model for predicting the set of GA progression parameters based on the comparison.

Embodiment 6. The method of any one of embodiments 1-5, further comprising: modifying a model of the plurality of models to form a new model based on the visualization output generated for the model to improve a performance of the model.

Embodiment 7. The method of any one of embodiments 1-6, wherein the set of GA progression parameters comprises at least one of a growth rate for the GA lesion or a baseline lesion area for the GA lesion.

Embodiment 8. The method of any one of embodiments 1-7, wherein the set of retinal images comprises at least one of a set of fundus autofluorescence (FAF) images or a set of optical coherence tomography (OCT) images.

Embodiment 9. The method of embodiment 8, wherein the set of fundus autofluorescence (FAF) images is a set of baseline FAF images and wherein the set of optical coherence tomography (OCT) images is a set of baseline OCT images.

Embodiment 10. A method for evaluating geographic atrophy in a retina, the method comprising: receiving a set of retinal images; predicting a set of geographic atrophy (GA) progression parameters for a geographic atrophy (GA) lesion in the retina using the set of retinal images and a deep learning model; and generating a set of gradient activation maps corresponding to the set of retinal images for the deep learning model, wherein a gradient activation map in the set of gradient activation maps for a corresponding retinal image of the set of retinal images identifies a set of regions in the corresponding retinal image that is relevant to predicting the set of GA progression parameters by the deep learning model.

Embodiment 11. The method of embodiment 10, further comprising: generating an output for use in improving a performance of the deep learning model based on the set of gradient activation maps.

Embodiment 12. The method of embodiment 10 or embodiment 11, wherein the set of GA progression parameters comprises at least one of a growth rate for the GA lesion or a baseline lesion area for the GA lesion.

Embodiment 13. The method of any one of embodiments 10-12, wherein the set of retinal images comprises at least one of a set of fundus autofluorescence (FAF) images or a set of optical coherence tomography (OCT) images.

Embodiment 14. A system for evaluating geographic atrophy, the system comprising: a memory containing machine readable medium comprising machine executable code; and a processor coupled to the memory, the processor configured to execute the machine executable code to cause the processor to: receive a set of retinal images; train each model of a plurality of models to predict a set of geographic atrophy (GA) progression parameters for a geographic atrophy (GA) lesion using the set of retinal images; and generate a visualization output for each model of the plurality of models, wherein the visualization output for a corresponding model of the plurality of models provides information about how the corresponding model uses the set of retinal images to predict the set of GA progression parameters.

Embodiment 15. The system of embodiment 14, wherein the visualization output includes a gradient activation map for a corresponding retinal image in the set of retinal images for the corresponding model, and wherein the gradient activation map indicates a set of regions in the corresponding retinal image that contributed to the set of GA progression parameters predicted by the corresponding model for the GA lesion.

Embodiment 16. The system of embodiment 14 or embodiment 15, wherein the corresponding model is a corresponding deep learning model and wherein the processor is configured to execute the machine executable code to cause the processor to validate the corresponding deep learning model using the visualization output generated for the corresponding deep learning model.

Embodiment 17. The system of any one of embodiments 14-16, wherein the plurality of models includes a first deep learning model and a second deep learning model and wherein the processor is configured to execute the machine executable code to cause the processor to: perform a comparison of the visualization output generated for the first deep learning model with the visualization output generated for the second deep learning model; and select either the first deep learning model or the second deep learning model as a best model for predicting the set of GA progression parameters based on the comparison.

Embodiment 18. The system of any one of embodiments 14-17, wherein the processor is configured to execute the machine executable code to cause the processor to modify a model of the plurality of models to form a new model based on the visualization output generated for the model to improve a performance of the model.

Embodiment 19. The system of any one of embodiments 14-18, wherein the set of GA progression parameters comprises at least one of a growth rate for the GA lesion or a baseline lesion area for the GA lesion.

Embodiment 20. The system of any one of embodiments 14-19, wherein the set of retinal images comprises at least one of a set of fundus autofluorescence (FAF) images or a set of optical coherence tomography (OCT) images.

### VIII. Additional Considerations

The headers and subheaders between sections and subsections of this document are included solely for the purpose of improving readability and do not imply that features cannot be combined across sections and subsection. Accordingly, sections and subsections do not describe separate embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The description provided herein provides preferred exemplary embodiments only, and is not intended to limit the scope, applicability or configuration of the disclosure. Rather, the description of the preferred exemplary embodiments will provide those skilled in the art with an enabling description for implementing various embodiments. It is understood that various changes may be made in the function and arrangement of elements (e.g., elements in block or schematic diagrams, elements in flow diagrams, etc.) without departing from the spirit and scope as set forth in the appended claims.

Specific details are given in the above description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

What is claimed is:

1. A method for evaluating geographic atrophy, the method comprising:
receiving a set of retinal images;
training each model of a plurality of models to predict a set of geographic atrophy (GA) progression parameters for a geographic atrophy (GA) lesion using the set of retinal images; and
generating a visualization output for each model of the plurality of models, wherein the visualization output for a corresponding model of the plurality of models provides information about how the corresponding model uses the set of retinal images to predict the set of GA progression parameters.

2. The method of claim 1, wherein the generating comprises:
generating a gradient activation map for a corresponding retinal image in the set of retinal images for the corresponding model, wherein the gradient activation map indicates a set of regions in the corresponding retinal image that contributed to the set of GA progression parameters predicted by the corresponding model for the GA lesion.

3. The method of claim 1, wherein the plurality of models includes a deep learning model and further comprising:
validating the deep learning model using the visualization output generated for the deep learning model.

4. The method of claim 1, wherein the plurality of models includes a first deep learning model and a second deep learning model further comprising:
performing a comparison of the visualization output generated for the first deep learning model with the visualization output generated for the second deep learning model.

5. The method of claim 4, further comprising:
selecting either the first deep learning model or the second deep learning model as a best model for predicting the set of GA progression parameters based on the comparison.

6. The method of claim 1, further comprising:
modifying a model of the plurality of models to form a new model based on the visualization output generated for the model to improve a performance of the model.

7. The method of claim 1, wherein the set of GA progression parameters comprises at least one of a growth rate for the GA lesion or a baseline lesion area for the GA lesion.

8. The method of claim 1, wherein the set of retinal images comprises at least one of a set of fundus autofluorescence (FAF) images or a set of optical coherence tomography (OCT) images.

9. The method of claim 8, wherein the set of fundus autofluorescence (FAF) images is a set of baseline FAF images and wherein the set of optical coherence tomography (OCT) images is a set of baseline OCT images.

10. A method for evaluating geographic atrophy in a retina, the method comprising:
receiving a set of retinal images;
predicting a set of geographic atrophy (GA) progression parameters for a geographic atrophy (GA) lesion in the retina using the set of retinal images and a deep learning model; and
generating a set of gradient activation maps corresponding to the set of retinal images for the deep learning model, wherein a gradient activation map in the set of gradient activation maps for a corresponding retinal image of the set of retinal images identifies a set of regions in the corresponding retinal image that is relevant to predicting the set of GA progression parameters by the deep learning model.

11. The method of claim 10, further comprising:
generating an output for use in improving a performance of the deep learning model based on the set of gradient activation maps.

12. The method of claim 10, wherein the set of GA progression parameters comprises at least one of a growth rate for the GA lesion or a baseline lesion area for the GA lesion.

13. The method of claim 10, wherein the set of retinal images comprises at least one of a set of fundus autofluorescence (FAF) images or a set of optical coherence tomography (OCT) images.

14. A system for evaluating geographic atrophy, the system comprising:
a memory containing machine readable medium comprising machine executable code; and
a processor coupled to the memory, the processor configured to execute the machine executable code to cause the processor to:
receive a set of retinal images;
train each model of a plurality of models to predict a set of geographic atrophy (GA) progression parameters for a geographic atrophy (GA) lesion using the set of retinal images; and
generate a visualization output for each model of the plurality of models, wherein the visualization output for a corresponding model of the plurality of models provides information about how the corresponding model uses the set of retinal images to predict the set of GA progression parameters.

15. The system of claim 14, wherein the visualization output includes a gradient activation map for a corresponding retinal image in the set of retinal images for the corresponding model, and wherein the gradient activation map indicates a set of regions in the corresponding retinal image that contributed to the set of GA progression parameters predicted by the corresponding model for the GA lesion.

16. The system of claim 14, wherein the corresponding model is a corresponding deep learning model and wherein the processor is configured to execute the machine executable code to cause the processor to validate the corresponding deep learning model using the visualization output generated for the corresponding deep learning model.

17. The system of claim 14, wherein the plurality of models includes a first deep learning model and a second deep learning model and wherein the processor is configured to execute the machine executable code to cause the processor to:
perform a comparison of the visualization output generated for the first deep learning model with the visualization output generated for the second deep learning model; and
select either the first deep learning model or the second deep learning model as a best model for predicting the set of GA progression parameters based on the comparison.

18. The system of claim 14, wherein the processor is configured to execute the machine executable code to cause the processor to modify a model of the plurality of models to form a new model based on the visualization output generated for the model to improve a performance of the model.

19. The system of claim 14, wherein the set of GA progression parameters comprises at least one of a growth rate for the GA lesion or a baseline lesion area for the GA lesion.

20. The system of claim 14, wherein the set of retinal images comprises at least one of a set of fundus autofluorescence (FAF) images or a set of optical coherence tomography (OCT) images.
